(19) United States
(12) Patent Application Publication
(10) Pub. No.: US 2024/0426854 A1
(43) Pub. Date: Dec. 26, 2024

(54) METHODS AND SYSTEMS FOR DETECTING AN ORIENTATION OF A CASSETTE

(71) Applicant: RAPID MICRO BIOSYSTEMS, INC., Lowell, MA (US)

(72) Inventors: Michael WILT, Lowell, MA (US); Celso SILVESTRE, Lowell, MA (US); Kyle HOWE, Lowell, MA (US); Peter TIA, Lowell, MA (US)

(21) Appl. No.: 18/747,382

(22) Filed: Jun. 18, 2024

Related U.S. Application Data
(60) Provisional application No. 63/509,141, filed on Jun. 20, 2023.

(51) Int. Cl. G01N 35/00; G01N 35/02

(52) U.S. Cl. CPC G01N 35/00732; G01N 35/00069; G01N 35/025; G01N 2035/00891

(57) ABSTRACT

Exemplary embodiments pertain to an automated sample processing system that includes a cassette having a superior component and an inferior component, where the superior component and the inferior component have a sufficient delta in opacity that is detectable by a sensor. An emitter is configured to emit a signal, where the signal passes laterally through one component of the cassette (or is diffused by it) and is configured to be received by a sensor. The sensor is configured to communicate the signal processing logic that is configured to interpret the signal and indicate the orientation of the cassette. Also disclosed is a method of detecting the orientation of a cassette in the automated sample processing system.

### CROSS-REFERENCE TO RELATED APPLICATIONS

This application claims priority to and the benefit of U.S. Provisional Application No. 63/509,141, filed Jun. 20, 2023, the entirety of which is incorporated herein by reference.

### BACKGROUND

Many industries require the determination of the number or presence of microbes in a sample, often referred to as microbe enumeration or detection. One method of determining the number of microbes in a sample (or detecting the presence of microbes) involves exposing a filtration membrane to a sample to capture microbes in the sample on the membrane, culturing the microbes captured on the membrane, and optionally counting the number of colonies that grow during culturing.

The membrane may be provided inside a cassette to protect the microbe culture during incubation and/or analysis. The cassette may also facilitate automated processing by holding and organizing samples as they move through various stages of processing. An example of an automated sample processing system is the GROWTH DIRECT™ system from RAPID MICRO BIOSYSTEMS™ of Lowell, Massachusetts.

An operator may load the cassettes into a carousel that allows multiple cassettes to be analyzed in an automated sample analysis system. The system may retrieve the cassettes from the carousel and perform an analysis on the samples in the cassette. During some types of automated sample analysis, illuminated samples are caused to fluoresce without destroying the samples. By combining digital imagining technology with sophisticated software algorithms, these systems can detect and count the autofluorescence of growing microbes.

The cassette holding the sample is thus designed with an optically clear or transparent lid, through which the light shines. The bottom of the cassette is typically opaque to block out any background light.

A common error when using systems for automated processing of cassettes is for operators to load cassettes into the system with the clear lid facing down. This is considered upside-down. This is a relatively common mistake, because conventional manual incubation is often performed with the clear lid facing down during incubation and technicians rely on old habits from the manual method. In other cases, there may be lapses in operator training. When the cassette is loaded in an upside-down configuration, the system cannot image the sample in the cassette and an operator must manually flip the cassette over before automated processing can occur. This can increase the amount of time required to analyze a sample, result in lost samples, delay the processing of other samples, and decrease system accuracy.

### SUMMARY

Exemplary embodiments provide a sensor system for use in automated sample processing systems. The sensor system implements a unique technique for detecting the orientation of each cassette before it is picked from the carousel and loaded into an incubator and/or imaged. Software logic monitors the sensor for an indication on the orientation of each cassette prior to the cassette being loaded into the incubator. When the sensor indicates the cassette is upside down, the software pauses loading to allow the cassette orientation to be corrected before restarting. The cassette orientation is corrected either manually by an operator, or automatically by the automated sample processing system. This prevents failures later in the process which could lead to lost samples and user dissatisfaction.

Disclosed is an automated sample processing system that includes a cassette having a superior component and an inferior component, where the superior component and the inferior component have a sufficient difference in opacity so as to be detectable by a sensor. The system also includes a carousel having a carousel base and at least one cassette column, where each cassette column includes at least one cassette. A cassette elevator is configured to raise and lower the carousel base longitudinally. An emitter is configured to emit a signal, where the signal passes laterally through the superior component of the cassette and is configured to be received by a sensor. The sensor is configured to communicate the signal to software logic on a computer, and the software logic is configured to interpret the signal and determine the orientation of the cassette.

Also disclosed is a method for detecting the orientation of a cassette in the automated sample processing system. The disclosed method includes loading a cassette having a superior component and an inferior component into a cassette column of a carousel, where the superior component and the inferior component are either combination of cassette lid and a cassette base having a sufficient delta in opacity that is detectable by a sensor, raising a carousel base of the carousel using an elevator, emitting a signal from an emitter, passing the signal laterally through the superior component of the cassette, receiving the signal by a sensor, communicating the signal result to software on a computer, and indicating the cassette orientation on a monitor connected to the computer. When the cassette lid is superior to the cassette base, the method may also further include the step of loading the cassette into an incubator as a result of a robust signal passing laterally through the cassette lid. When the cassette base is superior to the cassette lid, the method may also further include the step of stopping cassette loading into an incubator as a result of a non-robust signal passing laterally through the cassette base. In this case, the method may further include resuming cassette loading into the incubator when the cassette orientation is corrected automatically by the automated sample processing system or manually by an operator.

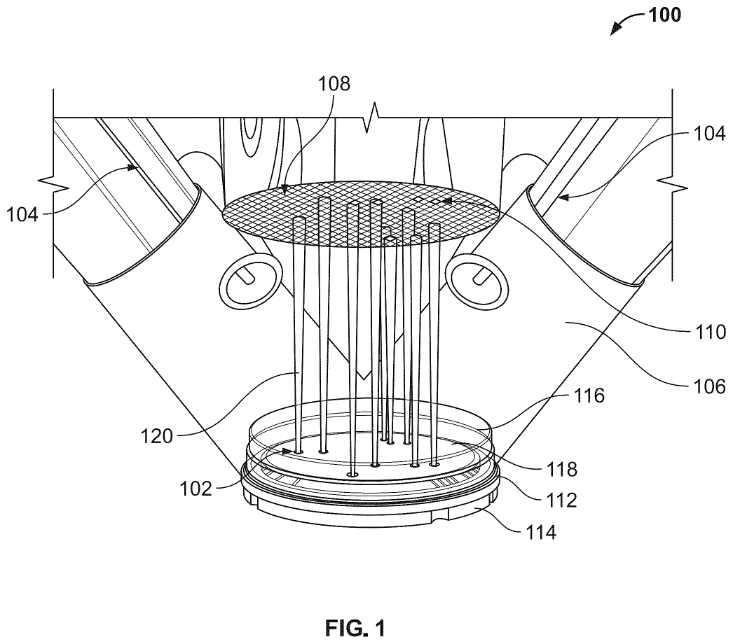

FIG. 1 illustrates an example of an automated sample analysis system, according to embodiments described herein.

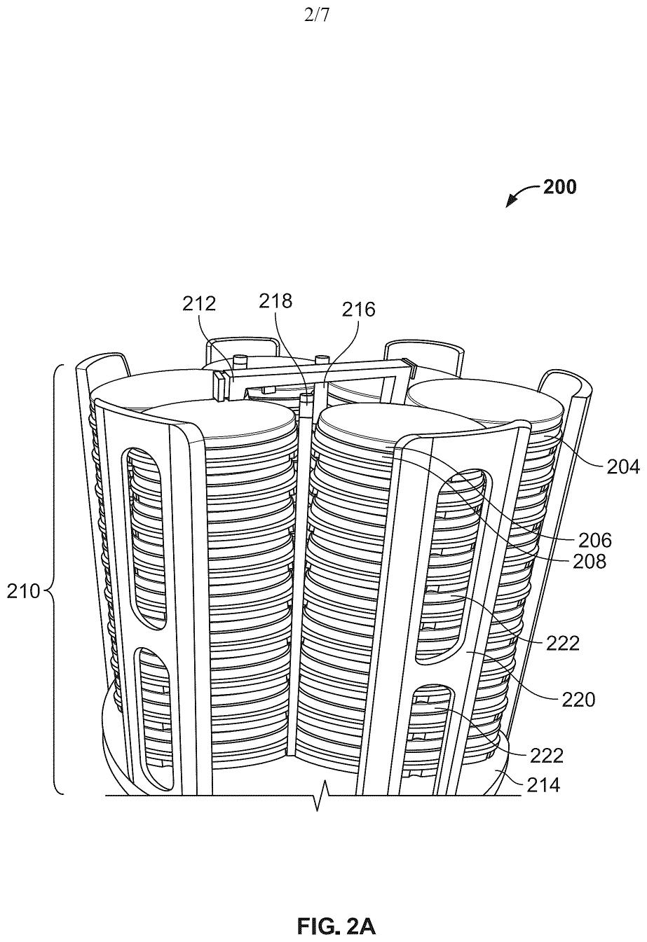

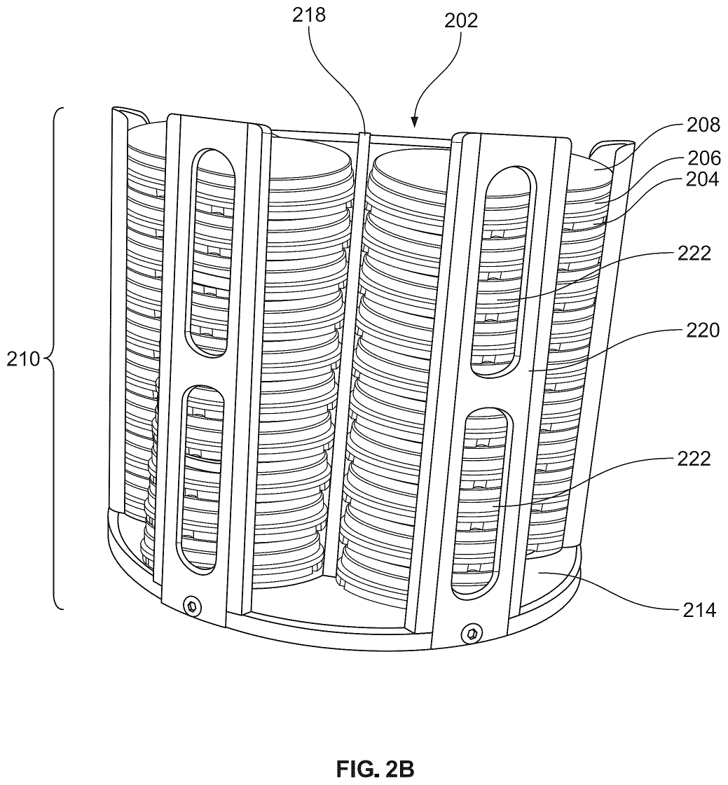

FIG. 2A illustrates an example of an incorrectly loaded cassette carousel, according to embodiments described herein.

FIG. 2B illustrates an example of a correctly loaded cassette carousel, in which each cassette is in a loaded position, according to embodiments described herein.

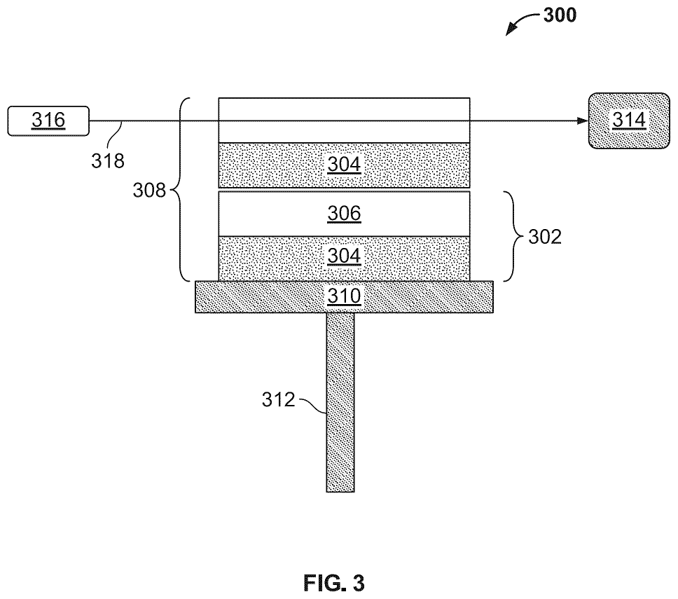

FIG. 3 is a block diagram of an automated sample processing system, according to embodiments described herein

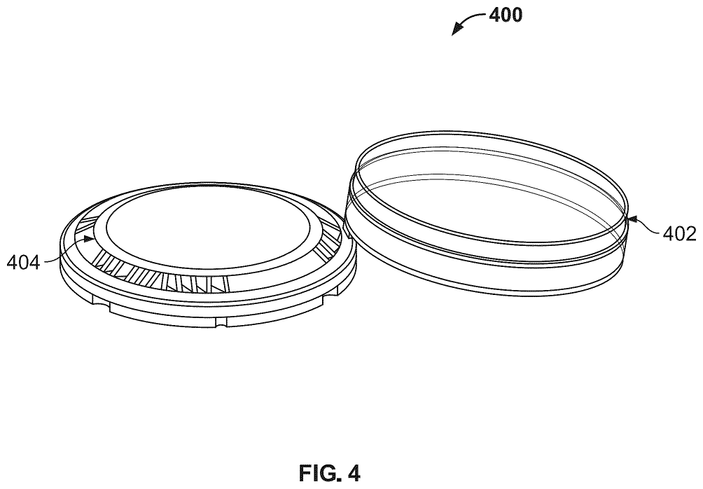

FIG. 4 illustrates an example of cassette components, according to embodiments described herein.

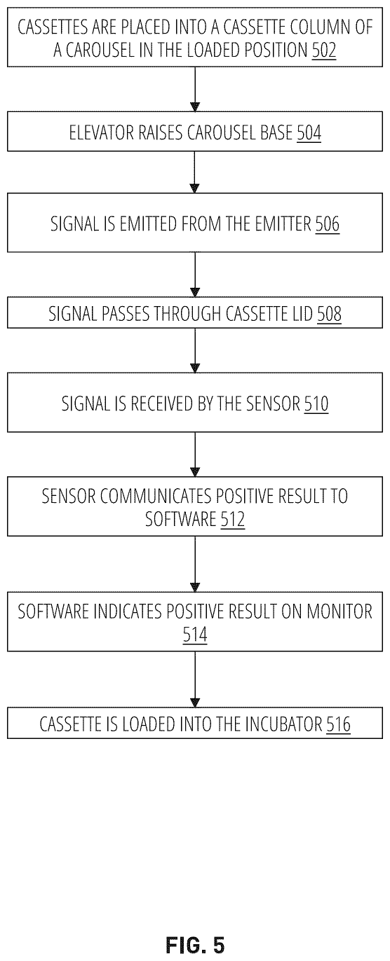

FIG. 5 illustrates an example method for loading cassettes into an incubator using an automated sample processing system in which the cassettes are in the correct loaded position, according to embodiments described herein.

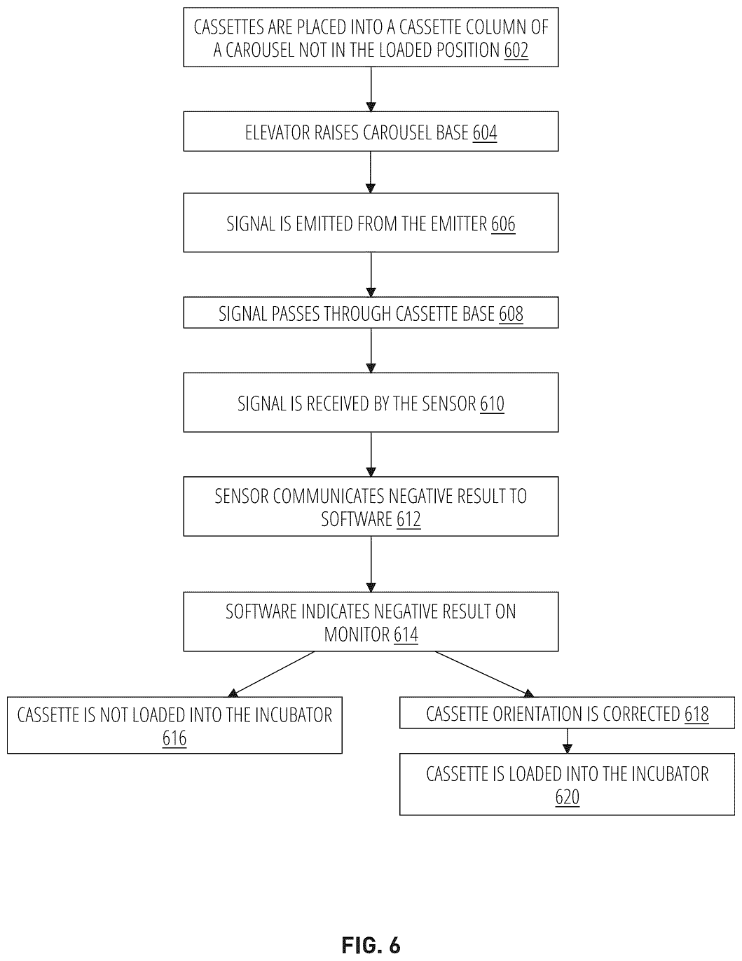

FIG. 6 illustrates an example method for loading cassettes into an incubator using an automated sample processing system in which the cassettes are not in the correct loaded position, according to embodiments described herein.

### DETAILED DESCRIPTION

### Automated Sample Analysis System

FIG. 1 illustrates an example of an automated sample analysis system 100, according to embodiments described herein. The analysis system 100 comprises microorganism colonies 102, Lamps 104 emitting light 106, a charge-couple device (CCD) chip 108, multiple photosensitive pixels 110, a cassette 112, a cassette base 114, a cassette lid 116, a growth media 118, and microorganism fluorescence 120.

In some embodiments, the CCD chip 108 comprises photosensitive pixels 110 to capture light emitted from the microorganisms 102.

In some embodiments, each cassette 112 comprises a cassette base 114 and a cassette lid 116. In some embodiments, the cassette base 114 contains growth media 118. In some embodiments, the growth media 118 contains microorganism colonies 102. In some embodiments, the microorganism colonies 102 in the growth media 118 are exposed to Lamp light 106 produced by the Lamps 104. In some embodiments, the microorganisms in the microorganism colonies 102 produces microorganism fluorescence 120. In some embodiments, the microorganism fluorescence 120 are captured by the photosensitive pixels 110 of the CCD chip 108.

In some embodiments, the analysis system 100 comprises one or more lamps 104. In some embodiments, the at least one lamp is a light emitting diode (LED) lamp, an incandescent lamp, a fluorescent lamp, a halogen lamp, a high-intensity discharge (HID) lamp, a neon lamp, a laser lamp, a visible light lamp, an ultraviolet (UV) lamp, an infrared (IR) lamp, or any other type of light source capable of producing light. In some embodiments, the sun produces light for the analysis system 100.

In some embodiments, the light 106 produced by the Lamps 104 is incandescent lamp light, fluorescent lamp light, halogen lamp light, high-intensity discharge (HID) lamp light, neon lamp light, laser lamp light, visible light, ultraviolet (UV) light, infrared (IR) light, or any other type of light.

In some embodiments, the analysis system 100 comprises at least one cassette 112. In some embodiments, the growth media 118 comprises one type of microorganism colony 102. In some embodiments, the growth media 118 comprises more than one type of microorganism colony 102. The microorganism colonies 102 may, when exposed to the light 106, produce microorganism fluorescence 120. In some embodiments, different types of microorganism colonies 102 may be distinguished by the different types of microorganism fluorescence 120 that each produces.

In some embodiments, the analysis system 100 comprises one or more CCD chips 108. The CCD chips 108 may include one or more photosensitive pixels 110. The photosensitive pixels 110 may convert the microorganism fluorescence 120 into electrical signals. In some embodiments, the electrical signals are interpreted by logic on the analysis system 100 to indicate the number of microorganism colonies 102 in the growth media 118. In some embodiments, an imaging device other than a CCD chip 108 may be used to capture the microorganism fluorescence 120. In some embodiments, the imaging device is a complementary metal-oxide-semiconductor (CMOS) image sensor, a specific type of CMOS image sensor (such as an active pixel sensor CMOS), a scientific CMOS (sCMOS) image sensor, an indium gallium arsenide (InGaAs) image sensor, other types of CCD chips (such as an electron-multiplying CCD chip), or any other type of sensor capable for imaging.

In some embodiments, the cassette lid 116 of the cassette 112 is substantially transparent. The cassette lid 116 may be considered substantially transparent when light emitted by a selected light source is capable of passing from one side of the cassette lid 116 to another such that the light remains perceptible by a sensor after passing through the cassette lid 116 from side to side. A substantially transparent cassette lid 116 may also allow the lamp light 106 to pass through the top of the cassette lid 116 to illuminate the microorganism colonies and/or to allow the fluorescence 120 to pass through the cassette lid 116 so that it remains perceptible by the photosensitive pixels 110. In some embodiments, the top surface of the cassette lid 116 may have a different optical property than the side surfaces of the cassette lid 116. The top surface may be configured to allow light from the lamps 104 and the fluorescence 120 to pass through, whereas the side surfaces may be configured to allow light from a side-mounted light source to pass through the cassette lid 116 and remain perceptible by a side-mounted sensor. The amount of transparency in the top surface may be selected based on the type of lamp 104 used, whereas the amount of transparency in the side surfaces may be selected based on the type or capabilities of side-mounted light source and/or side-mounted sensor.

In some embodiments, the cassette base 114 of the cassette 112 is substantially opaque. The cassette base may be considered substantially opaque when light from the side-mounted light source, which is capable of being perceived by the side-mounted sensor when it passes through the cassette lid 116, is not capable of being perceived by the side-mounted sensor when it passes through the cassette base 114. A substantially opaque cassette base 114 may also block background light from passing through the body of the cassette base 114.

### Automated Sample Processing System

FIG. 2A illustrates an example of an incorrectly loaded carousel 200, according to embodiments described herein. In some embodiments, the incorrectly loaded carousel 200 comprises cassettes 204 having cassette bases 206 and cassette lids 208, cassette columns 210, a carousel handle 212, a carousel base 214, a carousel stem 216, column posts 218, column supports 220, and column apertures 222.

In some embodiments, the cassettes 204 on top of each of the cassette columns 210 (the most superior cassettes 204 of each cassette column 210) of the incorrectly loaded carousel 200 are loaded upside down, with the cassette base 206 positioned superior to the cassette lid 208. Exemplary embodiments are capable of detecting the configuration of the cassettes when some, all, or none of the cassettes are loaded upside down, in any combination or order.

In some embodiments, an inferior end of the carousel stem 216 is coupled to the superior face of the carousel base 214. In some embodiments, the superior end of the carousel stem 216 is coupled to the carousel handle 212. In some embodiments, each cassette column 210 is coupled to the carousel base 214.

In some embodiments, the carousel stem 216 is substantially cylindrical. In some embodiments, the carousel stem 216 is substantially rectangular or any shape capable of coupling to the carousel base 214 and the carousel handle 212.

In some embodiments, the carousel handle 212 is T-shaped. In some embodiments, the carousel handle 212 is straight, D-shaped, a knob, a ring, or any shape capable to be gripped to lift the incorrectly loaded carousel 200. In some embodiments, the lateral ends of the carousel handle 212 extend longitudinally to couple to the carousel base 214, thereby forming an aperture between the longitudinally extended lateral end of the carousel handle 212 and the carousel stem 216.

In some embodiments, the carousel handle 212 is central to the incorrectly loaded carousel 200, surrounded by the at least one cassette column 210. In some embodiments, the carousel handle 212 is not in the center of the incorrectly loaded carousel 200.

In some embodiments, the carousel base 214 is substantially circular. In some embodiments, the carousel base 214 is substantially square, rectangular, triangular, pentagonal, hexagonal, heptagonal, or any other shape capable of holding at least one cassette column 210.

Each cassette column 210 may include zero or more column posts 218 and zero or more column supports 220. In some embodiments, the column support 220 comprises zero or more column apertures 222. The column apertures 222 may be evenly spaced or not evenly spaced. The column apertures 222 may be of any suitable shape, such as oval shaped, square, rectangular, circular, triangular, pentagonal, hexagonal, or any other shape.

In some embodiments, the column support 220 is substantially concave relative to the carousel stem 216. In some embodiments, the column support 220 is a substantially straight surface. In some embodiments, the column support 220 is substantially convex relative to the carousel stem 216.

In some embodiments, each column post 218 is positioned internally and adjacent to the carousel stem 216 and the column support 220 is positioned externally with the outer edge of the column support 220 coupled to the superior perimeter of the carousel base 214. In some embodiments, each column post 218 is positioned externally with the outer edge of the column post 218 coupled to the superior perimeter of the carousel base 214 and the column support 220 is positioned internally and adjacent to the carousel stem 216.

In some embodiments, each cassette column 210 shares at least one column post 218 with an adjacent cassette column 210. In some embodiments, each cassette column 210 shares both column posts 218 with the adjacent cassette columns 210. In some embodiments, each cassette column 210 comprises its own column posts 218. In some embodiments, the lateral ends of the carousel handle 212 couple to column posts 218.

In the depicted embodiments, the carousel 200 includes six cassette columns 210, although the present invention is not limited to this configuration. Each cassette column 210 may have a capacity for zero or more cassettes 112 (the specific number of cassettes being dependent on the size of the carousel 200 and the size of the cassettes 112).

FIG. 2B illustrates an example of a correctly loaded carousel 202, in which each cassette 204 is in a loaded position, according to embodiments described herein. The loaded position is in which the cassette lid 208 is positioned superior to the cassette base 206. In some embodiments, the incorrectly loaded carousel 200 comprises cassettes 204, the cassette bases 206, the cassette lids 208, the cassette columns 210, the carousel handle 212 (shown in FIG. 2A), the carousel base 214, the column carousel stem 216 (shown in FIG. 2A), the column posts 218, the column supports 220, and the column apertures 222.

In some embodiments, all of the cassettes 204 in each cassette column 210 are in the loaded position, with the cassette lid 208 positioned superior to the cassette base 206. In some embodiments, one or more cassettes 204 in one or more cassette columns 210 are loaded right-side up. In some embodiments, the cassettes 204 on the top of each cassette column 210 are loaded right-side up.

FIG. 3 illustrates a block diagram of an automated sample processing system 300, according to embodiments described herein. The system comprises two cassettes 302 as shown and described in FIG. 1-FIG. 4, each comprising a cassette base 304 and a cassette lid 306 as shown and described in FIG. 1-FIG. 4, a cassette column 308 as shown and described in FIG. 2A-FIG. 2B, a carousel base 310 as shown and described in FIG. 2A-FIG. 2B, a carousel elevator 312, a sensor 314, an emitter 316, and a signal 318.

In some embodiments, the cassette column 308 comprises one cassette 302. In some embodiments, the cassette column 308 comprises more than two cassettes 302. In some embodiments, the cassette column 308 has no cassettes 302.

In some embodiments, the emitter 316 is a light emitter. In some embodiments, the emitter 316 is a laser emitter. In some embodiments, the emitter 316 is an incandescent light emitter, a fluorescent light emitter, a LED emitter, a halogen light emitter, a high-intensity discharge (HID) light emitter, a neon light emitter, a visible light emitter, an ultraviolet (UV) light emitter, an infrared (IR) light emitter, or any other type of emitter that can produce light. In some embodiments, the sun is the emitter 316. In some embodiments, the emitter 316 is any type of heat emitter. In some embodiments, the emitter 316 is any type of device capable of producing any signal, including but not limited to an analog signal emitter, a digital signal emitter, an electromagnetic signal emitter, an audio signal emitter, a video signal emitter, or an optical signal emitter.

In some embodiments, the signal 318 is a form of light. In some embodiments, the signal 318 is a laser. In some embodiments, the signal 318 is incandescent light, fluorescent light, LED light, halogen light, high-intensity (HID) light, neon light, visible light, ultraviolet (UV) light, infrared (IR) light, or any other type of light. In some embodiments, sunlight is the signal 318. In some embodiments, the signal 318 is any type of heat. In some embodiments, the signal 318 is any form of indication that is produced by an emitter and received by a sensor, including but not limited to an analog signal, a digital signal, an electromagnetic signal, an audio signal, a video signal, a motion signal, or an optical signal.

In some embodiments, the sensor 314 is a light sensor. In some embodiments, the sensor 314 is a laser sensor. In some embodiments, the sensor 314 is an incandescent light sensor, a fluorescent light sensor, a LED sensor, a halogen light sensor, a high-intensity discharge (HID) light sensor, a neon light sensor, a visible light sensor, an ultraviolet (UV) light sensor, an infrared (IR) light sensor, or any other type of sensor that can receive light. In some embodiments, the sensor 314 is a solar panel. In some embodiments, the sensor 314 is any type of heat receiver. In some embodiments, the sensor 314 is any device capable of receiving any signal, including but not limited to an analog signal sensor, a digital signal sensor, an electromagnetic signal sensor, an audio signal sensor, a video signal sensor, a motion sensor, or an optical signal sensor.

In some embodiments, a vision system is used to analyze the cassette for a feature which is unique to the top or bottom of the cassette to enable the orientation of the cassette to be understood. In some embodiments, the vision system could detect a feature such as: the physical geometry of the cassette, fiducials built in to the cassette or applied via printer or labeling, barcodes etched, printed or labeled on the cassette, alphanumeric text etched, printed or labeled on the cassette or any other markings which provide a unique understanding of whether the top or bottom of the cassette is facing up.

In some embodiments, the carousel elevator 312 is a device that is capable of raising and lowering the carousel base 310 longitudinally. In some embodiments, the carousel elevator 312 raises the carousel base 310 longitudinally such that the cassette 302 on top of the cassette column 308 is in a loaded position. In the loaded position, the cassette 302 is positioned to allow the signal 318 produced by the emitter 316 to pass through the cassette lid 306 unobstructed for reception by the sensor 314.

In some embodiments, when the cassette 302 on top of the cassette column 308 is in the loaded position, the signal 318 passes from the emitter 316 to the sensor 314 with high strength, because the signal 318 passes through the substantially transparent cassette lid 306 which allows for most of the signal 318 to pass through. In some embodiments, if the cassette 302 is upside down, with the substantially opaque cassette base 304 positioned above the substantially transparent cassette lid 306 (as shown in FIG. 2A), the signal 318 will not pass through the substantially opaque cassette base 304, resulting in the signal detection being much lower or completely missing.

In some embodiments, in the loaded position, the signal 318 passes through the center latitudinal axis of the cassette lid 306. In some embodiments, in the loaded position, the signal 318 passes through the superior latitudinal half of the cassette lid 306 (i.e. between the center latitudinal axis of the cassette lid 306 and the superior face of the cassette lid 306). In some embodiments, in the loaded position the signal 318 passes through the inferior latitudinal half of the cassette lid 306 (i.e. between the center latitudinal axis of the cassette lid 306 and center latitudinal axis of the cassette 302). In some embodiments, in the loaded position, the signal 318 passes through the cassette lid 306 from the superior latitudinal half of the cassette lid 306 to the inferior latitudinal half of the cassette lid 306. In some embodiments, in the loaded position, the signal 318 passes through the cassette lid 306 from the inferior latitudinal half of the cassette lid 306 to the superior latitudinal half of the cassette lid 306.

In some embodiments, the emitter 316 and sensor 314 are positioned such that the signal 318 passes through the top component of the cassette 302 (i.e. the component positioned superior to the other). In some embodiments, in the loaded position, the signal 318 passes through the cassette lid 306 horizontally, with the emitter 316 and sensor 314 laterally level with each other. In some embodiments, in the loaded position, the signal 318 passes through the cassette lid 306 at an angle such that the emitter 316 is laterally above the sensor 314. In some embodiments, in the loaded position, the signal 318 passes through the cassette lid 306 at an angle such that the emitter 316 is laterally below the sensor 314.

Although embodiments are shown in which the emitter 316 emits a signal into the superior component of the cassette 302, it is also contemplated that the emitter 316 may emit a signal into the inferior component of the cassette 302. For example, if the emitter 316 is positioned so as to emit the signal into the inferior component of the cassette, the system may interpret the absence (or attenuation) of the signal as indicating that the cassette is in the right-side up configuration. If the sensor detects an unattenuated or robust signal, this may indicate that the cassette is upside down.

In some embodiments, the sensor and emitter need not be on opposite sides of the cassette. For instance, in some examples the emitter may emit a signal (such as light) into a superior component that is constructed of a material capable of diffusing the signal. The inferior component may not be capable of diffusing the signal, or may diffuse the signal to a different degree (such that the difference is distinguishable based on the sensor signal). The sensor may be located adjacent to the emitter, or anywhere else along the circumference of the cassette where it can register whether it is receiving a diffuse signal from the superior component or a non-diffuse signal.

Moreover, although exemplary embodiments are generally described in connection with a signal representing light, the present invention is not so limited. It is also contemplated that other types of signals may be used; any type of signal capable of being transmitted by an emitter and received by a sensor after passing through the cassette is suitable. The terms transparent and opaque may generally refer to materials that have the property of relaying, transmitting, or otherwise manipulating the signal into a first configuration and a second configuration, respectively. The first configuration may be the presence of the signal and the second configuration may be the absence of the signal. In other embodiments, the first configuration may present the signal to the sensor in an unmanipulated configuration and the second configuration may present the signal to the sensor in a manipulated configuration (e.g., filtered, attenuated, augmented, supplemented, with different properties, etc.) or vice versa.

In some embodiments, the sensor interprets the signal emitted by the emitter (as a result of passing through either the cassette lid 306 or the cassette base 304) and transmits the results to signal processing logic that is housed on a computer. In some embodiments, if the sensor receives a robust signal, the signal processing logic indicates a positive result on a monitor or display via the computer (or a negative result, if the signal processing logic does not receive a robust signal, or otherwise receives a signal indicating an upside down orientation of the cassette). In some embodiments, upon the signal processing logic receiving a positive result, the cassette is loaded into an incubator or an imaging system configured to capture an image of the interior of the cassette through a top surface of the cassette's superior component. In some embodiments, the cassette is automatically picked up by the automated sample processing system (e.g., using a robotic arm) from the cassette column of the carousel and loaded into the incubator. In some embodiments, the cassette is manually picked up by the operator from the cassette column of the carousel and loaded into the incubator.

In some embodiments, if the sensor does not receive a robust signal, the signal processing logic indicates a negative result on the monitor via the computer. In some embodiments, a prominent visual indicator and message is displayed on the monitor to the operator to inform the operator of the problem with the cassette orientation. In some embodiments, simultaneous with or in lieu of the visual indicator, the computer communicates an electronic notification message to pre-configured addresses in order to alert operators who may be remote.

In some embodiments, the cassette is not loaded into the incubator. In some embodiments, in the event that the cassette orientation is not corrected, loading of cassettes into the incubator stops until the orientation is corrected either by the operator or the automated sample processing system.

In some embodiments, the cassette orientation is corrected. In some embodiments, the cassette orientation is corrected manually by the operator. In some embodiments, the automated sample processing system may have the capability to automatically flip the cassettes into the correct orientation (e.g., through the use of a robotic arm). In some embodiments, the cassette is loaded into an incubator or imaging system. In some embodiments, the cassette is automatically picked up with a robotic arm from the cassette column of the carousel and loaded into the incubator or imaging system. In some embodiments, the cassette is manually picked up by the operator from the cassette column of the carousel and loaded into the incubator or imaging system.

FIG. 4 illustrates an example of cassette components, according to embodiments described herein. The cassette 400 comprises a cassette lid 402 and a cassette base 404. In this example, the cassette lid 402 and cassette base 404 are shown before they are coupled.

In some embodiments, any transparent plastic may be useful for the purpose of the substantially transparent cassette lid 402 of the cassette 400. In some embodiments, the cassette lid 402 may be made of an optically transparent non-fluorescent plastic. For the detection of upside-down cassettes 400, any plastic may be suitable to create the cassette lid 402, so long as it allows a sufficient amount of light (or other form of signal) to pass through for sensor analysis purposes. In some embodiments, any material may be suitable to create the cassette lid 402 so long as it allows a sufficient amount of light (or other form of signal) to pass through for analysis purposes, such as glass, acrylic, polycarbonate, polyethylene terephthalate (PET), ceramic, or quartz.

In some embodiments, “transparent” may mean allowing at least 85% of light to pass through, although the present invention is not so limited. In practice, the definition of a “transparent” cassette lid 306 may depend on the optical detection capabilities of the sensor 314 (shown in FIG. 3) used by the automated sample processing system-“transparent” may simply refer to a degree of transparency that does not attenuate the optical characteristics of the cassette 400 beyond a point that the sensor 314 (shown in FIG. 3) of the automated sample processing system can obtain an acceptable reading during sample processing. Alternatively, a cassette lid 402 may be considered “transparent” when the opacity of the cassette lid 402 is sufficiently different from the opacity of the cassette base 404 of the cassette 400 so that the emitter 316/sensor 314 pair (shown in FIG. 3) of the present embodiments can detect a difference between them.

In some embodiments, any non-transparent plastic may be useful for the purpose of the substantially opaque cassette base 404 of the cassette 400. In some embodiments, the cassette base 404 may be made of an optically opaque non-fluorescent plastic. For the detection of upside-down cassettes 400, any plastic may be suitable, as long as it blocks a sufficient amount of light from passing through for analysis purposes. In some embodiments, any material may be suitable to create the cassette base 404 so long as it blocks a sufficient amount of light from passing through for analysis purposes, such as metal, wood, brick, concrete, ceramic, or rubber.

In some embodiments, “opaque” may mean blocking at least 85% of light from passing through, although the present invention is not so limited. In practice, the definition of a “opaque” cassette base 404 may depend on the optical detection capabilities of the sensor 314 (shown in FIG. 3) used by the automated sample processing system-“opaque” may simply refer to a degree of opacity that does not attenuate the optical characteristics of the cassette 400 beyond a point that the sensor 314 (shown in FIG. 3) of the automated sample processing system can obtain an acceptable reading during sample processing. Alternatively, a cassette base 404 may be considered “opaque” when the opacity of the cassette base 404 is sufficiently different from the opacity of the cassette lid 402 so that the emitter 316/sensor 314 pair (shown in FIG. 3) of the present embodiments can detect a difference between them. The emitted signal 318 (shown in FIG. 3) should be attenuated enough by the plastic of the cassette base 404 that the sensor 314 (shown in FIG. 3) reading is sufficiently different from the reading of the less opaque plastic of the cassette lid 402.

In some embodiments, in practice, the cassette lid 402 and cassette base 404 of the cassette 400 do not need to be fully transparent and fully opaque, respectively. In some embodiments, the cassette lid 402 and the cassette base 404 comprise a sufficient delta of the signal (shown in FIG. 3) strength capable of being transmitted through the material to allow a robust detection. In some embodiments, the placement and calibration of the sensor (shown in FIG. 3) is such that the signal (shown in FIG. 3) received through the more transparent part of the cassette 400 (the cassette lid 402) is significantly different from the signal (shown in FIG. 3) received through the more opaque part of the cassette 400 (the cassette base 404). This difference in signal value allows software of the automated sample processing system, housed on a computer, to determine if the transparent portion of the cassette is on top versus the opaque portion. This information is used to determine if the cassette is upside down.

### Automated Sample Processing Method

In some embodiments, a method for using an automated sample processing system to detect the orientation of each cassette before it is picked up from the carousel and loaded in to an incubator is described herein. In some embodiments, a method for detecting the orientation of a cassette in an automated sample processing system includes loading the cassette having a superior component and an inferior component into the cassette column of the carousel, where the superior component and the inferior component are either combination of cassette lid and a cassette base having a sufficient delta in opacity that is detectable by the sensor, raising the carousel base of the carousel using the elevator, emitting the signal from the emitter, passing the signal laterally through the superior component of the cassette, receiving the signal by the sensor, communicating the signal result to software on the computer, and indicating the cassette orientation on the monitor connected to the computer. When the cassette lid is superior to the cassette base, the method may also further include the step of loading the cassette into an incubator as a result of a robust signal passing laterally through the cassette lid. When the cassette base is superior to the cassette lid, the method may also further include the step of stopping cassette loading into an incubator as a result of a non-robust signal passing laterally through the cassette base. In this case, the method may further include resuming cassette loading into the incubator when the cassette orientation is corrected automatically by the automated sample processing system or manually by an operator.

FIG. 5 illustrates an example method for loading cassettes into an incubator or image processing system using an automated sample processing system in which the cassettes are in the correct loaded position, according to embodiments described herein. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

In step 502, the cassettes (shown in FIG. 2B) are placed into a cassette column (shown in FIG. 2B) of a carousel (shown in FIG. 2B) in the loaded position. In some embodiments, the cassettes are automatically placed into a cassette column using a robotic arm. In some embodiments, the cassettes are manually placed into a cassette column by an operator.

In step 504, the elevator (shown in FIG. 3) raises the carousel base (shown in FIG. 2B).

In step 506, the signal (shown in FIG. 3) is emitted from the emitter (shown in FIG. 3).

In step 508, the signal passes through the cassette lid (shown in FIG. 2B). In some embodiments, prior to the cassette being picked up from the carousel to be loaded into an incubator (not shown), the automated sample processing system uses an emitter/sensor (shown in FIG. 3) pair to send and receive, respectively, a signal intended to pass though the cassette lid (shown in FIG. 3). In some embodiments, the cassette lid and the cassette base (shown in FIG. 3) comprise a sufficient delta of the signal strength capable of being transmitted through the material.

In step 510, the signal is received by the sensor (shown in FIG. 3). In some embodiments, the cassette lid allows for a robust detection of the signal, while the cassette base does not allow for a robust detection of the signal. This difference in signal value allows the automated sample processing system to determine if the transparent portion of the cassette is on top versus the opaque portion. This information is used to determine if the cassette is upside down. In this embodiment, the cassette lid is right side up.

In step 512, the sensor communicates the positive result to a software (not shown) on a computer. In some embodiments, robust detection of the signal is communicated via the sensor to the software, indicating that the transparent portion of the cassette is on top versus the opaque portion. The cassette lid 402 and the cassette base 404 comprise a sufficient delta of the signal strength capable of being transmitted through the material to allow a robust detection.

In step 514, the software indicates the positive result on a monitor via a computer. In some embodiments, the monitor is viewed by an operator.

In step 516, the cassette is loaded into an incubator or image processing system. In some embodiments, the cassette is automatically picked up with a robotic arm from the cassette column of the carousel and loaded into the incubator or image processing system. In some embodiments, the cassette is manually picked up by the operator from the cassette column of the carousel and loaded into the incubator or image processing system.

FIG. 6 illustrates an example method for loading cassettes into an incubator or image processing system using an automated sample processing system in which the cassettes are not in the correct loaded position, according to embodiments described herein. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

In step 602, the cassettes (shown in FIG. 2A) are placed into a cassette column (shown in FIG. 2A) of a carousel (shown in FIG. 2A) not in the loaded position (i.e. the cassettes are upside down). In some embodiments, the cassettes are automatically placed into a cassette column using a robotic arm. In some embodiments, the cassettes are manually placed into a cassette column by an operator.

In step 604, the elevator (shown in FIG. 3) raises the carousel base (shown in FIG. 2A).

In step 606, the signal (shown in FIG. 3) is emitted from the emitter (shown in FIG. 3).

In step 608, the signal passes through the cassette base (shown in FIG. 2A). In some embodiments, prior to the cassette being picked up from the carousel to be loaded into an incubator (not shown), the automated sample processing system uses an emitter/sensor (shown in FIG. 3) pair to send and receive, respectively, a signal intended to pass though the cassette lid (shown in FIG. 3). In some embodiments, such as in step 608, the cassette is loaded incorrectly with the cassette base positioned superior as opposed to the cassette lid. In some embodiments, the cassette lid and the cassette base (shown in FIG. 3) comprise a sufficient delta of the signal strength capable of being transmitted through the material.

In step 610, the signal is received by the sensor. In some embodiments, the cassette base does not allow for a robust detection of the signal, while the cassette lid does allow for a robust detection of the signal. This difference in signal value allows the automated sample processing system to determine if the transparent portion of the cassette is on top versus the opaque portion. This information is used to determine if the cassette is upside down. In this embodiment, the cassette base is right side up. In some embodiments, the signal is not received by the sensor.

In step 612, the sensor communicates the negative result to a software (not shown) on a computer. In some embodiments, non-robust detection of the signal is communicated via the sensor to the software, indicating that the opaque portion of the cassette is on top versus the transparent portion. The cassette lid 402 and the cassette base 404 comprise a sufficient delta of the signal strength capable of being transmitted through the material to allow a robust detection.

In step 614, the software indicates the negative result on a monitor via a computer. In some embodiments, the monitor is viewed by an operator. In some embodiments, a prominent visual indicator and message is displayed on the monitor to the operator to inform the operator of the problem with the cassette orientation. In some embodiments, simultaneous with or in lieu of the prominent visual indicator, the computer communicates an electronic notification message to pre-configured addresses in order to alert operators who may be remote.

In step 616, the cassette is not loaded into the incubator or image processing system. In some embodiments, in the event that the cassette orientation is not corrected, loading of cassettes into the incubator or image processing system stops until the orientation is corrected either by the operator or the automated sample processing system (e.g., the use of a robotic arm).

In step 618, the cassette orientation is corrected. In some embodiments, the cassette orientation is corrected manually by the operator. In some embodiments, the automated sample processing system may have the capability to automatically flip the cassettes into the correct orientation (e.g., through the use of a robotic arm).

In step 620, the cassette is loaded into an incubator or image processing system. In some embodiments, the cassette is automatically picked up with a robotic arm or image processing system from the cassette column of the carousel and loaded into the incubator. In some embodiments, the cassette is manually picked up by the operator from the cassette column of the carousel and loaded into the incubator.

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one non-transitory computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, the manipulations performed are often referred to in terms, such as adding or comparing, which are commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general purpose computer as selectively activated or reconfigured by a computer program stored in the computer. The procedures presented herein are not inherently related to a particular computer or other apparatus. Various general purpose machines may be used with programs written in accordance with the teachings herein, or it may prove convenient to construct more specialized apparatus to perform the required method steps. The required structure for a variety of these machines will appear from the description given.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

Exemplary embodiments include, but are not limited to the following:

1. An automated sample processing system, comprising: a cassette comprising a superior component having a lateral side and an inferior component having a lateral side, wherein the lateral side of the superior component and the lateral side of the inferior component have different opacities; a carousel configured to support the cassette; an emitter configured to emit a signal into a lateral side of the superior component of the cassette; a sensor capable of receiving the signal at least when the signal passes through a cassette component that is more optically transparent than the other cassette component; and signal processing logic operable on a processor, the sensor configured to communicate an output to the signal processing logic, the signal having a first characteristic when passing through the more optically transparent cassette component and a second characteristic when passing through the other cassette component, the signal processing logic configured to interpret the output to determine an orientation of the cassette.

2. The automated sample processing system of 1, wherein the first characteristic is the presence of the signal and the second characteristic is the absence of the signal.

3. The automated sample processing system of any of 1 or 2, wherein the signal processing logic is further configured to display the orientation of the cassette on a display.

4. The automated sample processing system of any of 1-3, wherein the emitter and sensor are arranged so that, when the carousel positions the cassette in a predetermined configuration, the emitter and sensor are configured to be adjacent to the lateral side of the superior component on opposite sides of the cassette.

5. The automated sample processing system of any of 1-4, wherein the emitter and sensor are arranged so that, when the carousel positions the cassette in a predetermined configuration, the emitter and sensor are configured to be adjacent to the lateral side of the inferior component on opposite sides of the cassette.

6. The automated sample processing system of any of 1-5, wherein the signal is light of a predetermined wavelength and the sensor is a photodetector.

7. The automated sample processing system of any of 1-6, wherein the superior component is one of a cassette lid or a cassette base, and the inferior component is the other of the cassette lid or the cassette base.

8. The automated sample processing system of 7, wherein the cassette lid is substantially transparent to the signal emitted by the emitter and the cassette base is substantially opaque to the signal emitted by the emitter.

9. The automated sample processing system of any of 7-8, wherein the cassette lid is comprised of an optically-clear non-fluorescent plastic.

10. The automated sample processing system of any of 7-9, wherein cassette base is comprised of a substantially opaque plastic.

11. The automated sample processing system of any of 1-10, further comprising a robotic manipulator configured to flip the cassette to place the inferior component in a superior position, and further comprising control logic configured to instruct the robotic manipulator to flip the cassette when the signal processing logic determines that the cassette is in an upside-down orientation.

12. The automated sample processing system of any of 1-11, further comprising control logic configured to perform one or more of: pausing automatic processing of the cassette when the signal processing logic determines that the cassette is in an upside-down orientation, and/or automatically processing the cassette when the signal processing logic determines that the cassette is in a right-side up orientation, or

13. The automated sample processing system of any of 1-12, further comprising an imaging system configured to capture an image through a top surface of the superior component of the cassette, wherein the signal processing logic is configured to determine the orientation of the cassette before the cassette is presented to the imaging system.

14. An automated sample processing system, comprising: a cassette comprising a superior component having a lateral side and an inferior component having a lateral side, wherein the lateral side of the superior component and the lateral side of the inferior component have different diffusion properties; a carousel configured to support the cassette;
an emitter configured to emit a signal into a lateral side of either the superior component or the inferior component of the cassette; a sensor capable of receiving the signal at least when the signal passes is diffused by a cassette component that has a greater capability to diffuse the signal than the other cassette component; and signal processing logic operable on a processor, the sensor configured to communicate an output to the signal processing logic, the signal having a first characteristic when passing through the cassette component with the greater capability to diffuse the signal and a second characteristic when passing through the other cassette component, the signal processing logic configured to interpret the output to determine an orientation of the cassette.

15. A method for detecting the orientation of a cassette in an automated sample processing system, comprising: loading a cassette into a carousel, the cassette comprising a superior component having a lateral side and an inferior component having a lateral side, wherein the lateral side of the superior component and the lateral side of the inferior component have different opacities; emitting a signal into a lateral side of the superior component of the cassette using an emitter, the signal capable of being received by a sensor at least when the signal passes through a cassette component that is more optically transparent than the other cassette component; and processing an output of the sensor using signal processing logic, the signal having a first characteristic when passing through the more optically transparent cassette component and a second characteristic when passing through the other cassette component, the signal processing logic configured to interpret the output to determine an orientation of the cassette.

16. The method of 15, wherein the first characteristic is the presence of the signal and the second characteristic is the absence of the signal.

17. The method of any of 15-16, further comprising instructing a robotic manipulator to flip the cassette when the signal processing logic determines that the cassette is in an upside-down orientation.

18. The method of any of 15-17, further comprising pausing automatic processing of the cassette when the signal processing logic determines that the cassette is in an upside-down orientation.

19. The method of any of 15-18, further comprising automatically processing the cassette when the signal processing logic determines that the cassette is in a right-side up orientation.

20. The method of any of 15-20, wherein determining the orientation of the cassette is performed before the cassette is presented to an imaging system configured to capture an image through a top surface of the superior component of the cassette.

What is claimed is:

1. An automated sample processing system, comprising:
a cassette comprising a superior component having a lateral side and an inferior component having a lateral side, wherein the lateral side of the superior component and the lateral side of the inferior component have different opacities;
a carousel configured to support the cassette;
an emitter configured to emit a signal into a lateral side of either the superior component or the inferior component of the cassette;
a sensor capable of receiving the signal at least when the signal passes through a cassette component that is more optically transparent than the other cassette component; and
signal processing logic operable on a processor, the sensor configured to communicate an output to the signal processing logic, the signal having a first characteristic when passing through the more optically transparent cassette component and a second characteristic when passing through the other cassette component, the signal processing logic configured to interpret the output to determine an orientation of the cassette.

2. The automated sample processing system of claim 1, wherein the first characteristic is the presence of the signal and the second characteristic is the absence of the signal.

3. The automated sample processing system of claim 1, wherein the signal processing logic is further configured to display the orientation of the cassette on a display.

4. The automated sample processing system of claim 1, wherein the emitter and sensor are arranged so that, when the carousel positions the cassette in a predetermined configuration, the emitter and sensor are configured to be adjacent to the lateral side of the superior component on opposite sides of the cassette.

5. The automated sample processing system of claim 1, wherein the emitter and sensor are arranged so that, when the carousel positions the cassette in a predetermined configuration, the emitter and sensor are configured to be adjacent to the lateral side of the inferior component on opposite sides of the cassette.

6. The automated sample processing system of claim 1, wherein the signal is light of a predetermined wavelength and the sensor is a photodetector.

7. The automated sample processing system of claim 1, wherein the superior component is one of a cassette lid or a cassette base, and the inferior component is the other of the cassette lid or the cassette base.

8. The automated sample processing system of claim 7, wherein the cassette lid is substantially transparent to the signal emitted by the emitter and the cassette base is substantially opaque to the signal emitted by the emitter.

9. The automated sample processing system of claim 7, wherein the cassette lid is comprised of an optically-clear non-fluorescent plastic.

10. The automated sample processing system of claim 7, wherein cassette base is comprised of a substantially opaque plastic.

11. The automated sample processing system of claim 1, further comprising a robotic manipulator configured to flip the cassette to place the inferior component in a superior position, and further comprising control logic configured to instruct the robotic manipulator to flip the cassette when the signal processing logic determines that the cassette is in an upside-down orientation.

12. The automated sample processing system of claim 1, further comprising control logic configured to perform at least one of:
pausing automatic processing of the cassette when the signal processing logic determines that the cassette is in an upside-down orientation; or
automatically processing the cassette when the signal processing logic determines that the cassette is in a rightside-up orientation.

13. The automated sample processing system of claim 1, further comprising an imaging system configured to capture an image through a top surface of the superior component of the cassette, wherein the signal processing logic is configured to determine the orientation of the cassette before the cassette is presented to the imaging system.

14. An automated sample processing system, comprising:
a cassette comprising a superior component having a lateral side and an inferior component having a lateral side, wherein the lateral side of the superior component and the lateral side of the inferior component have different diffusion properties;
a carousel configured to support the cassette;
an emitter configured to emit a signal into a lateral side of either the superior component or the inferior component of the cassette;
a sensor capable of receiving the signal at least when the signal passes is diffused by a cassette component that has a greater capability to diffuse the signal than the other cassette component; and
signal processing logic operable on a processor, the sensor configured to communicate an output to the signal processing logic, the signal having a first characteristic when passing through the cassette component with the greater capability to diffuse the signal and a second characteristic when passing through the other cassette component, the signal processing logic configured to interpret the output to determine an orientation of the cassette.

15. A method for detecting the orientation of a cassette in an automated sample processing system, comprising:
loading a cassette into a carousel, the cassette comprising a superior component having a lateral side and an inferior component having a lateral side, wherein the lateral side of the superior component and the lateral side of the inferior component have different opacities;
emitting a signal into a lateral side of the superior component of the cassette using an emitter, the signal capable of being received by a sensor at least when the signal passes through a cassette component that is more optically transparent than the other cassette component; and
processing an output of the sensor using signal processing logic, the signal having a first characteristic when passing through the more optically transparent cassette component and a second characteristic when passing through the other cassette component, the signal processing logic configured to interpret the output to determine an orientation of the cassette.

16. The method of claim 15, wherein the first characteristic is the presence of the signal and the second characteristic is the absence of the signal.

17. The method of claim 15, further comprising instructing a robotic manipulator to flip the cassette when the signal processing logic determines that the cassette is in an upside-down orientation.

18. The method of claim 15, further comprising pausing automatic processing of the cassette when the signal processing logic determines that the cassette is in an upside-down orientation.

19. The method of claim 15, further comprising automatically processing the cassette when the signal processing logic determines that the cassette is in a right-side up orientation.

20. The method of claim 15, wherein determining the orientation of the cassette is performed before the cassette is presented to an imaging system configured to capture an image through a top surface of the superior component of the cassette.
